(19) United States
(12) Patent Application Publication
(10) Pub. No.: US 2012/0116432 A1
(43) Pub. Date: May 10, 2012

(54) TISSUE SECTIONING BLADE

(75) Inventor: Jared M. Orrock, Rochester, MN (US)

(73) Assignee: Mayo Foundation for Medical Education and Research, Rochester, MN (US)

(21) Appl. No.: 13/254,378

(22) PCT Filed: Mar. 4, 2010
(86) PCT No.: PCT/US2010/026214

§ 371 Date: Nov. 14, 2011

Related U.S. Application Data
(60) Provisional application No. 61/157,420, filed on Mar. 4, 2009.

(51) Int. Cl. A61B 17/3209

(52) U.S. Cl. 606/167

(57) ABSTRACT

A cutting device or tissue sectioning blade is disclosed that includes a blade body including a straight cutting edge, a back edge located opposite the straight cutting edge, a first end extending from the back edge to the straight cutting edge, and a second end located opposite the first end and extending from the back edge to the straight cutting edge.

### RELATED APPLICATION

The present application claims the benefit under 35 U.S.C. §119(e) of U.S. Provisional Application Ser. No. 61/157,420, titled TISSUE SECTIONING BLADE, filed on Mar. 4, 2009, which is incorporated herein by reference in its entirety.

The present invention relates to a medical device (e.g., a sectioning blade) for use in cutting sections of, for example, biological material.

Many types of medical devices (e.g., cutting tools) may be used in a wide variety of industries (e.g., the medical industry). For example, sectioning blades (e.g., disposable 130 mm and 260 mm sectioning blades) may be standard equipment in surgical pathology laboratories and autopsy departments around the world. Sectioning blades may be long razor blades, similar to straight razors used for shaving men's beards.

In a pathology lab, for example, sectioning blades may be used for cutting tissue specimens into thin slices or sections in preparation for microscopic evaluation. In some cases, sectioning blades are disposable, meaning that they are used and then disposed.

A tissue section may have any suitable thickness. For example, a tissue section may have approximately the same thickness as an American five-cent coin, which is about 2 millimeters thick. Cutting a tissue specimen to this thickness can be difficult, especially if the cutting surface or edge of the blade is not exquisitely sharp. Further, for cutting high quality sections, the specimen should be cut with as few strokes as possible, preferably cutting in one continuous movement. Each change in the direction of the blade may leave a ridge in the tissue that may distort or may cause uneven fixation of the tissue. Such distortion and uneven fixation may ultimately distort the microscopic appearance of the tissue specimen after a slide containing the tissue specimen is prepared.

The shape of the sectioning blade may be a rectangle. Thus, there may be two sharp corners on the cutting surface of the blade. As one is sectioning through a piece of tissue, both the leading (distal relative to a blade handle) and the trailing (proximal to a blade handle) edge or corner may catch (e.g., snag, etc.) on the specimen, a towel, or a table on which the specimen is resting. When an edge or corner catches, the cutter may be required to reverse the cutting direction, thereby creating a ridge in the tissue and decreasing the quality of the specimen.

### SUMMARY

The present disclosure provides a cutting device (e.g., a sectioning blade) that includes a blade body including a straight cutting edge, a back edge located opposite the straight cutting edge, a first end extending from the back edge to the straight cutting edge, and a second end located opposite the first end and extending from the back edge to the straight cutting edge. The first end may include a first curved segment proximate the straight cutting edge and a first end segment proximate the back edge. The first curved segment and the first end segment may meet at a location intermediate the straight cutting edge and the back edge.

The second end may include a second curved segment proximate the straight cutting edge and a second end segment proximate the back edge. The second curved segment and the second end segment may meet at a location intermediate the straight cutting edge and the back edge.

In some embodiments, the second end segment may be straight and/or may extend from the back edge to the second curved segment. In some embodiments, the second curved segment may extend from the second end segment to the straight cutting edge.

In some embodiments, the first end segment may be straight and/or may extend from the back edge to the first curved segment. In some embodiments, the first curved segment may extend from the first end segment to the straight cutting edge.

In one or more embodiments, the back edge may be generally aligned with the straight cutting edge. In some embodiments, the first end segment may be generally aligned with the second end segment. In some embodiments, one or both of the first and second end segments may be transverse to the straight cutting edge.

In one or more embodiments, the first end segment length may be at least about one-half of the distance between the straight cutting edge and the back edge at the first end. In one or more embodiments, the second end segment length may be at least about one-half of the distance between the straight cutting edge and the back edge at the second end.

In some embodiments, one or both of the first and second curved segments may be round and/or one or both of the first and second curved segments may enclose an angle of at most about ninety degrees.

In one or more embodiments, the first and second curved segments may be first and second curved cutting edges.

In some embodiments, one or both of the first and second end segments may be adjacent to the back edge and/or one or both of the first and second curved segments may be adjacent to the straight cutting edge.

In some embodiments, the first and second curved segments may be contiguous with the straight cutting edge.

In one or more embodiments, the blade body may be about 130 millimeters or about 260 millimeters from the first end to the second end. In some embodiments, the blade body may be about 20 millimeters from the back edge to the straight cutting edge.

In some embodiments, the blade body may be for sectioning tissue specimens (e.g., biological tissue specimens).

In one or more embodiments, the blade body may further include one or more holes and an optional blade handle may include one or more posts extending through the one or more holes of the blade body when removably attached to the blade body.

In some embodiments, the first end may be a leading end and the second end may be a trailing end and/or the first end may be a trailing end and the second end may be a leading end.

In a first aspect, some embodiments of the sectioning blades described herein include a blade body comprising a straight cutting edge, a back edge located opposite the straight cutting edge, a first end extending from the back edge to the straight cutting edge, and a second end located opposite the first end and extending from the back edge to the straight cutting edge; wherein the first end comprises a first curved segment proximate the straight cutting edge and a first end segment proximate the back edge, wherein the first curved segment and the first end segment meet at a location intermediate the straight cutting edge and the back edge such that the first curved segment and the first end segment extend from the straight cutting edge to the back edge of the blade body; and wherein the second end comprises a second curved segment proximate the straight cutting edge and a second end segment proximate the back edge, wherein the second curved segment and the second end segment meet at a location intermediate the straight cutting edge and the back edge such that the second curved segment and the second end segment extend from the straight cutting edge to the back edge of the blade body; and further wherein the first curved segment comprises a curved cutting edge.

In some embodiments of the first aspect, the second curved segment comprises a curved cutting edge.

In some embodiments of the first aspect, the first end segment is straight and wherein the second end segment is straight.

In some embodiments of the first aspect, the back edge is generally aligned with the straight cutting edge.

In some embodiments of the first aspect, the first end segment is straight and the second end segment is straight, and wherein the back edge is generally aligned with the straight cutting edge.

In some embodiments of the first aspect, the first end segment is generally aligned with the second end segment, and wherein the first and second end segments are generally transverse to the straight cutting edge.

In some embodiments of the first aspect, the first end segment length is at least about one-half of the distance between the straight cutting edge and the back edge at the first end.

In some embodiments of the first aspect, the first end segment length is at least about one-half of the distance between the straight cutting edge and the back edge at the first end, and wherein the second end segment length is at least about one-half of the distance between the straight cutting edge and the back edge at the second end.

In some embodiments of the first aspect, the first and second curved segments are extend over an arc of at most about ninety degrees.

In some embodiments of the first aspect, the first and second curved segments are contiguous with the straight cutting edge.

In some embodiments of the first aspect, the blade body comprises one or more holes and a blade handle, wherein the blade handle comprises one or more posts extending through the one or more holes of the blade body when attached to the blade body.

The words “preferred” and “preferably” refer to embodiments that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

As used herein and in the appended claims, the singular forms “a,” “an,” and “the” include plural references unless explicitly limited to the singular form or the context clearly dictates otherwise.

The term “and/or” means one or all of the listed elements or a combination of any two or more of the listed elements.

The above summary is not intended to describe each embodiment or every implementation of the present disclosure. Rather, a more complete understanding of the disclosure will become apparent and appreciated by reference to the following Description of Illustrative Embodiments and claims in view of the accompanying figures of the drawing.

### BRIEF DESCRIPTION OF THE FIGURES OF THE DRAWING

FIG. 1 is a plan view of a sectioning blade according to the present disclosure.

FIG. 2A is a plan view of a sectioning blade according to the present disclosure.

FIG. 2B is an enlarged perspective view of a portion of the sectioning blade of FIG. 2A.

FIGS. 3 and 4 are plan views of a sectioning blade according to the present disclosure.

FIGS. 5-7 are plan views a sectioning blade showing various sharpening options according to the present disclosure.

FIG. 8 is a plan view of a sectioning blade according to the present disclosure.

### DESCRIPTION OF ILLUSTRATIVE EMBODIMENTS

In the following description of illustrative embodiments, reference is made to the accompanying figures of the drawing which form a part hereof, and in which are shown, by way of illustration, specific embodiments of the sectioning blades described herein. It is to be understood that other embodiments may be utilized and structural changes may be made without departing from the scope of the present invention.

The present disclosure provides a cutting device (e.g., a sectioning blade). Although the cutting devices of the present disclosure may be used as a sectioning blade to, for example, cut biological tissue sections, the cutting devices of the present disclosure may also be utilized in a wide variety of procedures, medical and otherwise. Further, the cutting devices of the present disclosure may be utilized in pathology, histology, in autopsies, and other analyses of biological specimens.

Properly cutting sections may be challenging due to, for example, limitations of conventional equipment. For example, some sectioning blades may include sharp corners which may catch on the specimen, on a surface supporting the specimen, or on some material (e.g., fabric, a paper, etc.) that may be located between the specimen and the supporting surface. When the sectioning blade catches, the user of the sectioning blade may need to reverse the direction of the cutting motion, but may in the process create a ridge in the specimen that may reduce the quality of the specimen and the diagnoses drawn therefrom.

The sectioning blades of the present disclosure may, for example, reduce the number of changes in cutting direction, reduce the number of ridges in a tissue specimen section, improve the quality of the tissue specimen section, increase speed and efficiency of the sectioning process, and/or decrease the likelihood of inadvertent cuts to an individual using the cutting device.

One aspect of the present disclosure provides a sectioning blade that includes a blade body. The blade body may include a straight cutting edge, a back edge, a first end, and a second end. The back edge may be located opposite the straight cutting edge. The first end may extend from the back edge to the straight cutting edge. The second end may be located opposite the first end and may extend from the back edge to the straight cutting edge.

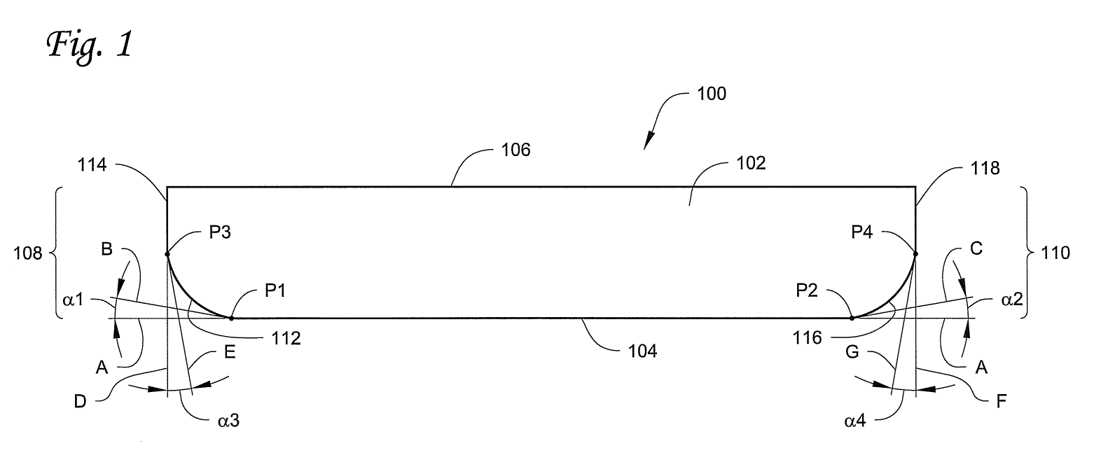

FIG. 1 illustrates the perimeter of a side view of at least one embodiment of the present disclosure. A sectioning blade 100 includes a blade body 102 that includes a straight cutting edge 104, a back edge 106, a first end 108 and a second end 110. The perimeter of a sectioning blade of the present disclosure, when viewed from at least one side may include a back edge 104, a first end 108, a straight cutting edge 104, and a second end 110.

In one or more embodiments of the present disclosure, at least a portion of the straight cutting edge may be generally straight (e.g., generally linear). Preferably, the straight cutting edge may be straight from the first end to the second end. In one or more embodiments, the length of the straight cutting edge may be any suitable length relative to the distance between the first end and the second end (e.g., the overall length of the blade body). For example, the straight cutting edge may be at least 10 percent of the distance from the first end to the second end. In some embodiments, the length of the straight cutting edge may be at least 25 percent, at least 50 percent, at least 75 percent, at least 90 percent, at least 95 percent, or at least 99 percent of the distance from the first end to the second end.

The straight cutting edge is an edge that has been sufficiently sharpened such that the sectioning blade may be used to cut a section of a biological specimen. The straight cutting edge may include a sharp edge along at least a portion of or, preferably, the entire length of the straight cutting edge. The blade body may include, for example, one or more bevels on one or both sides of the blade body proximal to the straight cutting edge to form a sufficiently sharp edge.

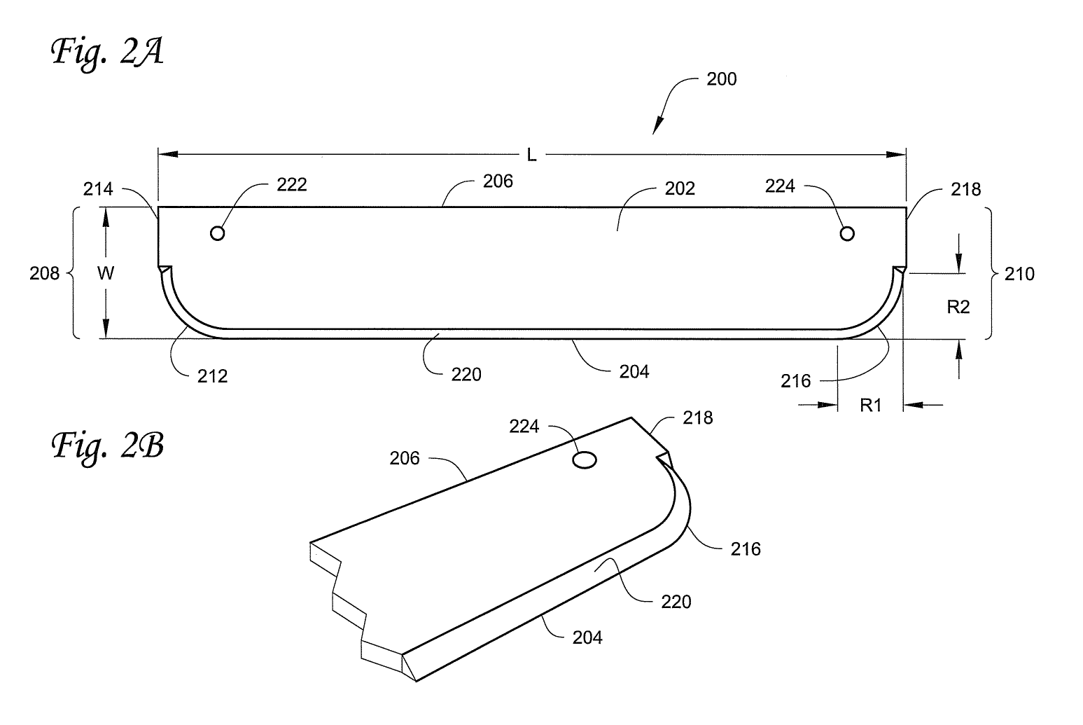

FIG. 2A shows a perspective view of a sectioning blade according to the present disclosure. The sectioning blade 200 includes a blade body 202 having a back edge 206, a straight cutting edge 204, a first end 208 having a first curved segment 212 and a first end segment 214, and a second end 210 having a second curved segment 216 and a second end segment 218. The blade body 202 includes one or more bevels 220 on one side of the sectioning blade 200 proximal to the straight cutting edge 204. In FIG. 2A, both of the first and second curved segments 212 and 216 are circular and are sharpened over their entire lengths (i.e., from the straight cutting edge 204 to the first end segment 214 and from the straight cutting edge 204 to the second end segment 218). Some dimensions of the blade body are shown in FIG. 2A. For example, the length of the blade body L, the width of the blade body W, and the dimensions of the second curved portion R1 and R2 are shown. In the embodiment shown in FIG. 2A, L is 130 to 260 millimeters, W is 20 millimeters, and R1 and R2 are both 10 millimeters (leaving a radius of curvature of 10 millimeters at the corner).

The blade body of the sectioning blade in FIG. 2A also includes two holes, 222 and 224. Each of these holes 222 and 224 may be configured to receive at least a portion of a handle (e.g., a post connected to a handle).

FIG. 2B shows one of the ends of the sectioning blade shown in FIG. 2A.

With further reference to FIG. 1, in one or more embodiments, the first end 108 includes a first curved segment 112 proximate the straight cutting edge 104 and a first end segment 114 proximate the back edge 106, wherein the first curved segment 112 and the first end segment 114 meet at a location intermediate the straight cutting edge 104 and the back edge 106.

In some embodiments, the first end segment may be straight and may extend from the back edge to the first curved segment. Further, the first curved segment may extend from the first end segment to the straight cutting edge. In FIG. 1, the first end segment 114 is straight and extends from the back edge 106 to the first curved segment 112. Also in FIG. 1, the first curved segment 112 extends from the first end segment 114 to the straight cutting edge 104. In some embodiments, the first end segment is not straight.

In the present disclosure, the first curved segment may include any suitable smooth curve. In the present disclosure, the first curved segment may be circular, elliptical, or non-circular and non-elliptical. In the present disclosure, the first curved segment may have one radius of curvature or may have more than one radius of curvature. In one or more embodiments of the present disclosure, the smooth curve may include one or more straight portions. For example, in one or more embodiments wherein a smooth curve includes two or more adjacent straight portions, the straight portions may differ in direction by less than 5 degrees, less than 2 degrees, less than 1 degree, or less than 0.1 degrees. For example, in one or more embodiments wherein a smooth curve includes one or more straight portions, a straight portion may be adjacent to one or more curved portions, wherein the straight portion smoothly transitions into the one or more curved portions.

In one or more embodiments, a sectioning blade 100 of the present disclosure includes a second end 110 that may include a second curved segment 116 proximate the straight cutting edge 104 and a second end segment 118 proximate the back edge 106, wherein the second curved segment 116 and the second end segment 118 meet at a location intermediate the straight cutting edge 104 and the back edge 106.

In one or more embodiments, the second end segment 118 may extend from the back edge 106 to the second curved segment 116 and the second curved segment 116 may extend from the second end segment 118 to the straight cutting edge 104.

In one or more embodiments of the present disclosure, the first and second curved segments may be contiguous with the straight cutting edge. In other words, the blade body includes a smooth transition between the first and second curved segments and the straight cutting edge. For example, the first and second curved segments may have a radius of curvature (where they meet the straight cutting edge) of at least about 4 millimeters or more, 8 millimeters or more, or even 10 millimeters or more.

In one or more embodiments of the present disclosure, the straight cutting edge 104 may define a straight cutting edge axis A and the first curved segment 112 may have a first tangent axis B that is tangent to the first curved segment 112 at a first meeting point P1 where the first curved segment 112 meets the straight cutting edge 104. The first tangent axis B may intersect with the straight cutting edge axis A, forming a first minor angle α1 that may be at most 20 degrees.

In one or more embodiments, the first minor angle α1 formed by the straight cutting edge axis A and the first tangent axis B may be at most 10 degrees, at most 5 degrees, at most 2 degrees, or at most 1 degree.

In some embodiments, the second curved segment 116 may have a second tangent axis C that is tangent to the second curved segment 116 at a second meeting point P2 where the second curved segment 116 meets the straight cutting edge 104.

The second tangent axis C may intersect with the straight cutting edge axis A, forming a second minor angle α2 that may be at most 20 degrees. In one or more embodiments, the second minor angle α2 formed by the straight cutting edge axis A and the second tangent axis C may be at most 10 degrees, at most 5 degrees, at most 2 degrees, or at most 1 degree. In one or more embodiments, the first and/or second minor angles α1, α2 formed by the straight cutting edge axis A and the first and/or second tangent axes B, C may be about zero degrees (e.g., the straight cutting edge axis A and the first and/or second tangent axes B, C may be generally aligned or may be the same axis).

In one or more embodiments of the present disclosure, the first end segment 114 may define a first end axis D and the first curved segment 112 may have a third tangent axis E that is tangent to the first curved segment 112 at a third meeting point P3 where the first curved segment 112 meets the first end segment 114. The third tangent axis E may intersect with the first end axis D, framing a third minor angle α3 that may be at most 20 degrees.

In one or more embodiments, the third minor angle α3 formed by the first end axis D and the third tangent axis E may be at most 10 degrees, at most 5 degrees, at most 2 degrees, or at most 1 degree.

In one or more embodiments, the third minor angle α3 formed by the first end axis D and the third tangent axis E may be about zero degrees (e.g., the first end axis D and the third tangent axis E may be generally aligned or may be the same axis).

In some embodiments, the second end segment 118 may define a second end axis F and the second curved segment 116 may have a fourth tangent axis G that is tangent to the second curved segment 116 at a fourth meeting point P4 where the second curved segment 116 meets the second end segment 118. The fourth tangent axis G may intersect with the second end axis F, forming a fourth minor angle α4 that may be at most 20 degrees. In one or more embodiments, the fourth minor angle α4 formed by the second end axis F and the fourth tangent axis G may be at most 10 degrees, at most 5 degrees, at most 2 degrees, or at most 1 degree. In one or more embodiments, the fourth minor angle α4 formed by the second end axis F and the fourth tangent axis G may be about zero degrees (e.g., the second end axis F and the fourth tangent axis G may be generally aligned or may be the same axis).

In one or more embodiments, the first end segment may define a first end axis that is tangent to the first curved segment at the point where the first curved segment meets the first end segment. In one or more embodiments, the second end segment defines a second end axis that is tangent to the second curved segment at the point where the second curved segment meets the second end segment.

In one or more embodiments, the straight cutting edge axis may be generally transverse to the first end axis and/or the second end axis (e.g., one or both of the first and second end segments may be generally transverse to the straight cutting edge) and/or may be generally aligned with the back edge (e.g., the straight cutting edge may be generally aligned with the back edge). In some embodiments, the first end segment may be generally aligned with the second end segment.

In one or more embodiments wherein the second end includes a second curved segment, the second curved segment may have some characteristics (e.g., shape, contour, sharpness, etc.) similar to those described in the present disclosure associated with the first curved segment. For example, in FIG. 1, the sectioning blade 100 includes a blade body 102 having a first end 108 that includes first curved segment 112 and a second end 110 that includes a second curved segment 116, wherein the first and second curved segments 112 and 116 both have the same radius of curvature.

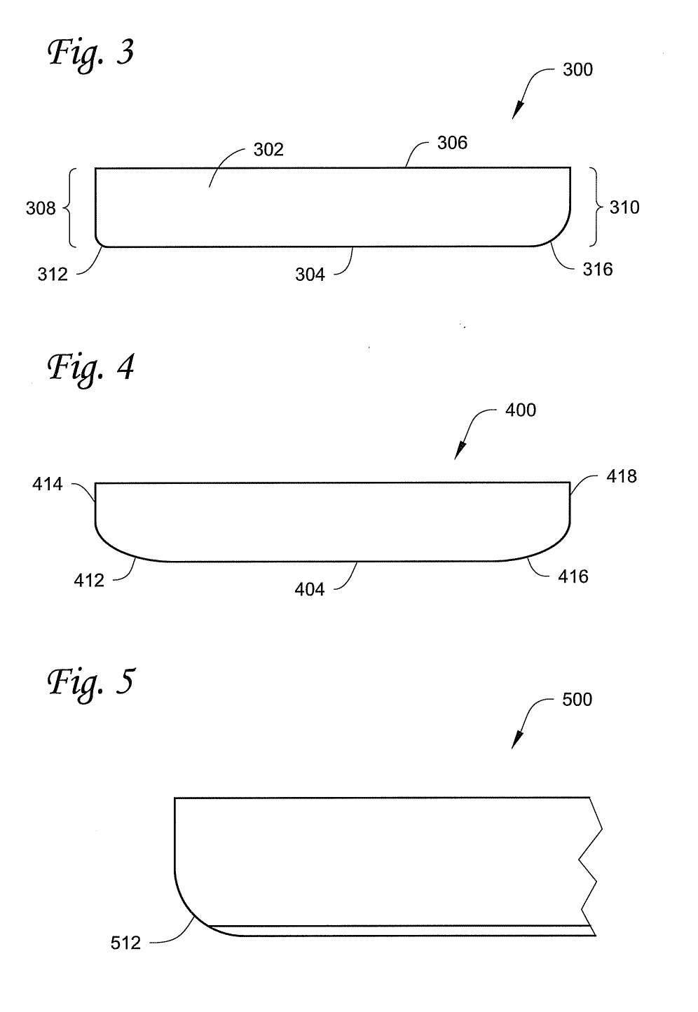

In the present disclosure, a wide variety of variations of the characteristics of the first and second curved segments are possible. For example, in FIG. 3, a sectioning blade 300 includes a blade body 302 that includes a back edge 306, a straight cutting edge 304, first end 308 having a first curved segment 312 having one radius of curvature, and a second end 310 having a second curved segment 316 that has a different radius of curvature.

FIG. 4 shows a sectioning blade 400 according to the present disclosure. In FIG. 4, the first and second curved segments 412 and 416 are elliptical (e.g., non-circular). In some embodiments, the first and second curved segments may be generally symmetrical (e.g., the shape or contour of one curved segment may be a mirror image of the other curved segment).

In one or more embodiments, the first curved segment may include more than one radius of curvature. For example, in FIG. 4, the first curved segment 412 includes a relatively short radius of curvature proximal the first end segment 414 and a relatively long radius of curvature proximal the straight cutting edge 404. In some embodiments, the radius of curvature of the first curved segment 412 may increase (or, alternatively, decrease) gradually from the first end segment 414 to the straight cutting edge 404.

Although a straight cutting edge may facilitate sectioning of tissue, in some embodiments, the straight cutting edge may, in reality, have a slight curvature, but that curvature should be sufficiently shallow such that the curve approximates a straight line.

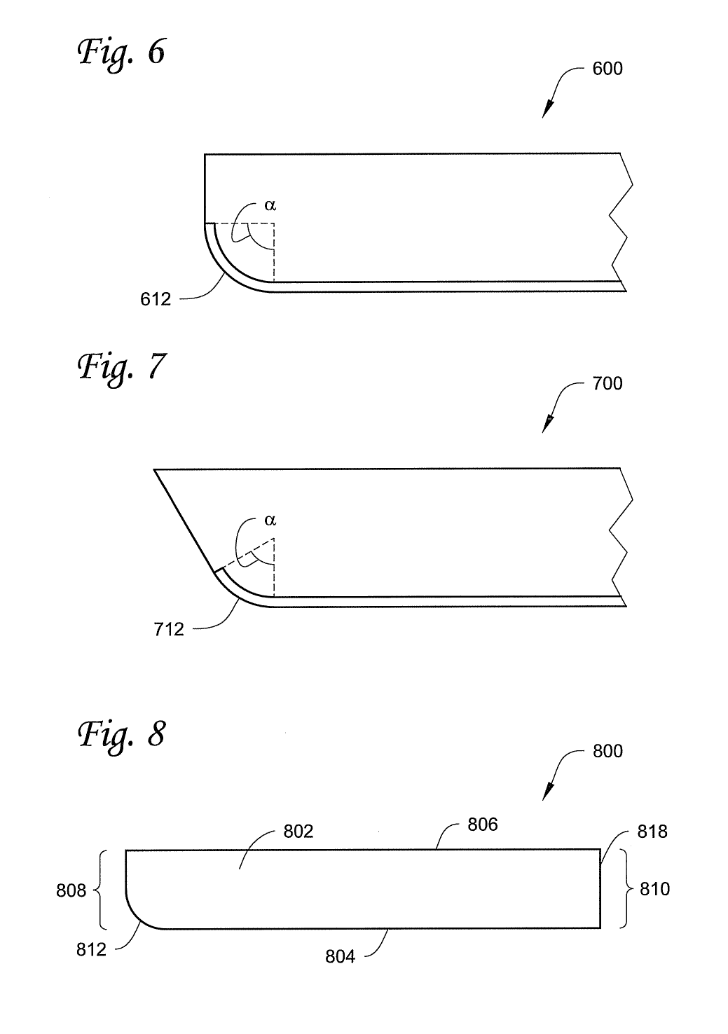

In some embodiments, one or both of the first and second curved segments may be sharpened. For example, FIG. 5 shows a portion of a sectioning blade 500 having a first curved segment 512, wherein some or all of the first curved segment 512 is not sharpened. In some embodiments, such as that shown in FIG. 6, the first curved segment 612 of a sectioning blade 600 may enclose an angle α of about ninety degrees, wherein all of the first curved segment 612 is sharpened (although leaving some portions of the curved segments unsharpened may, e.g., facilitate loading of the blade in a carrier).

Sectioning blades of the present disclosure may also include other configurations. For example, in FIG. 7, sectioning blade 700 includes a first curved portion 712 encloses an angle α of less than ninety degrees (e.g., 5, 10, 20, 30, 45, 60, 70, 80, or 85 degrees) and is sharpened. Other possible configurations include first curved segments that enclose any suitable angle and may or may not be sharpened. For example, in FIGS. 1-6, the first curved segments (112, 212, 312, 412, 512, and 612) enclose an angle α of about 90 degrees. In some embodiments, the angle enclosed by the first curved segment is at most 90 degrees. In one or more embodiments of the present disclosure, the angle enclosed by the second curved segment and the amount of the second curved segment that is sharpened may be the same or different than that of the first curved segment.

In one or more embodiments, other variations and combinations of shapes, radii of curvature, and degree of sharpness of the first and second curved segments may be possible.

In some embodiments in which the first and/or second curved segments are partially or entirely sharpened, the first and/or second curved segments may be first and second curved cutting edges. For example, FIGS. 6 and 7 show first curved segments (612 and 712) that are first cutting edges by virtue of being sharpened.

In one or more embodiments, the second end may include a second end segment, but may not include a second curved segment. For example, in FIG. 8, a sectioning blade 800 includes a blade body 802 that includes a back edge 806, a straight cutting edge 804, first end 808 having a first curved segment 812 (e.g. a first curved segment 812 that is circular, having one radius of curvature) and a second end 810 having a second end segment 818 that meets the straight cutting edge 804 at a corner.

In some embodiments, the first and/or second ends may include varying fractions of first and/or second end segments. A sectioning blade having at least a portion of one or both ends that is unsharpened may allow handling the blade body more safely. In one or more embodiments, the first end segment length may be at least about one-half of the distance between the straight cutting edge and the back edge at the first end. Similarly, in one or more embodiments, the second end segment length may be at least about one-half of the distance between the straight cutting edge and the back edge at the second end. For example, in FIG. 2A, the width W of the blade body is 20 millimeters at the first end 208 and the second end 210, and the first and second end segments 214 and 218 are each 10 millimeters, 10 millimeters being one half of the distance between the straight cutting edge 204 and the back edge 206 at each end.

In the present disclosure any suitable length and any suitable width of a blade body may be used. In some embodiments, the length of the blade body (i.e., the distance from the first end to the second end) may be about 130 millimeters or about 260 millimeters. Other lengths either between 130 millimeters and 260 millimeters or outside of this range may be used. In one or more embodiments, the width of the blade body (i.e., from the back edge to the straight cutting edge) may be about 20 millimeters, but may be any suitable width other than 20 millimeters.

In all of the embodiments described herein, the first end may be a leading end (i.e., the end of the blade body distal to an optional blade handle) and the second end may be a trailing end (e.g., the end of the blade body proximal to an optional blade handle). Alternatively, in all of the embodiments described herein, the first end may be a trailing end (i.e., the end of the blade body proximal to an optional blade handle) and the second end may be a leading end (e.g., the end of the blade body distal to an optional blade handle). In some embodiments, the blade body does not have sidedness (i.e., a blade body may be secured to a sectioning blade handle with the first end proximal to or distal to the sectioning blade handle).

In embodiments of the present disclosure, a blade body may include any suitable material or materials (e.g., metals, non-metals, alloys, ceramics, etc.) of construction. For example, the materials of construction of a blade body of the present disclosure may include stainless steel. A wide variety of materials of construction may be apparent to one of skill in the art.

In one or more embodiments, a blade body may be made by cutting or otherwise modifying the corners at one or both ends of the cutting edge of a conventional sectioning blade. In some embodiments, the sharp corners of a conventional sectioning blade may be rounded by any suitable machining techniques (e.g., cutting, filing, sharpening, etc.) such that the sectioning blade includes a first and/or second curved segment proximal to a straight cutting edge. In one or more embodiments, the procedures to make a conventional sectioning blade may be modified, such that the shape of the sectioning blade includes a first and/or second curved segment prior to sharpening. In such embodiments, the first and/or second curved segments may be sharpened along with the straight cutting edge.

In one or more embodiments, a blade body may be used to cut biological material (e.g., to section biological material for further analysis). In one or more embodiments, the blade body may be capable of cutting a tissue section having any suitable thickness. For example, a tissue section, in some embodiments, may be at most 5 millimeters, at most 4 millimeters, at most 3 millimeters, at most 2 millimeters, or may be at most 1 millimeter. In one or more embodiments, a tissue section may be greater than 5 millimeters thick.

A blade body of the present disclosure may include securing elements (e.g., holes, notches, ridges, etc.) that may be secured by an optional handle apparatus. A handle apparatus may include a handle portion operably connected to a securing portion. Preferably the securing elements of the blade body are configured to receive the securing portion of the handle apparatus, such that the blade body may be handled by a user (e.g., without actually touching the blade body).

A wide variety of securing elements may be used in the present disclosure. For each of the wide variety of securing elements, one or more configurations of securing portions of sectioning blade handles may be apparent to one skilled in the art. In the present disclosure a handle apparatus includes an apparatus for handling by a person, or an apparatus for handling by a machine, or any other suitable apparatus for handling the blade body.

In one or more embodiments, a sectioning blade may include a blade body having one or more holes and a handle apparatus (e.g., a blade handle) that includes one or more posts extending through the one or more holes of the blade body when the handle apparatus is removably attached to the blade body.

The following provides further description of one or more embodiments of the present disclosure:

A sectioning blade may be created that has rounded edges on the cutting surfaces.

In one or more embodiments of the devices of the present disclosure a rounded blade might not catch on a specimen or the surface below a specimen and may therefore allow for fewer changes in blade direction while cutting. This would result in sections with smoother surfaces (less ridging) and consequently better histology, which may be important for rendering an accurate diagnosis.

One or more devices of the present disclosure may make it easier to cut through large specimens and may improve the speed and efficiency at which a specimen could be sectioned, because a blade with rounded edges may not catch and the cutter may better be able to use the complete cutting surface of the blade.

Devices of the present disclosure may provide a heightened level of safety than associated with traditional sectioning blades. For example, because the blade may not catch, there may be fewer readjustments of the blade and/or tissue. A reduced number of adjustments of the blade and/or tissue may decrease the likelihood of inadvertent cuts to the individual using the blade.

By rounding the edges of the sectioning blade, a modification of an ubiquitous instrument in a surgical pathology and autopsy lab, an improvement in the quality of the tissue section, improved efficiency in sectioning a specimen, and/or reduced risk of injury may be realized.

Standard 130 millimeter and 260 millimeter surgical grade dissection blades may be constructed according to, for example, one or more embodiments described herein.

In one or more embodiments, the leading edge corner (the edge furthest from a handle on the cutting side of the blade) has a curve that follows a line that can be traced from a circle with a radius from about 1 millimeter to about 10 millimeters that is centered the same distance as its radius (from about 1 millimeter to about 10 millimeters) above the cutting edge and the same distance as the radius (from about 1 millimeter to about 10 millimeters) from the leading edge of the blade. In such embodiments, the leading edge of the blade may not be sharpened more than half way (up to 10 millimeters) up the leading edge. This may allow for at least 10 millimeters of the leading edge not to be sharpened, thereby making the blade easier to handle and load into the handle.

In one or more embodiments, the contour of the leading edge corner has a variable radius length, with the radius decreasing in length as it sweeps from the leading edge of the blade to the cutting surface.

In one or more embodiments, the leading edge corner is not sharpened at all. However, the leading edge corner of the blade still includes a rounded contour as described in the present disclosure. This rounded contour may allow for improved safety in handling of the blade at the expense of the ability of the leading edge to cut tissue. Because the corner is still rounded, the corner may not catch on the specimen or the surface below the specimen.

In one or more embodiments, the trailing edge corner (the edge closest to a handle on the cutting side of the blade) has a curve that follows a line that can be traced from a circle with a radius from about 1 millimeter to about 10 millimeters that is centered the same distance as its radius (from about 1 millimeter to about 10 millimeters) above the cutting edge and the same distance as the radius (from about 1 millimeter to about 10 millimeters) from the trailing edge of the blade. In such embodiments, the leading edge of the blade may not be sharpened more than half way (up to 10 millimeters) up the trailing edge. This may allow for at least 10 millimeters of the trailing edge not to be sharpened, thereby making the blade easier to handle and load into the handle.

In one or more embodiments, the contour of the trailing edge corner has a variable radius length, with the radius decreasing in length as it sweeps from the trailing edge of the blade to the cutting surface.

In one or more embodiments, the trailing edge corner is not sharpened at all. However, the trailing edge corner of the blade still includes a rounded contour as described in the present disclosure. This rounded contour may allow for improved safety in handling of the blade at the expense of the ability of the leading edge to cut tissue. Because the corner is still rounded, the corner may not catch on the specimen or the surface below the specimen.

In one or more embodiments, the blade body may have no sidedness. In other words, the shapes of the leading edge and the trailing edge may be the same, very similar, or symmetrical, which may facilitate loading of the blade body onto a handle because it may fit equally well, no matter how it was loaded (e.g., whether the first end was the trailing end or the leading end, etc.). In some embodiments, a sectioning blade may include shapes of the leading edge and trailing edge that may not be the same, may not be similar, or may not be symmetrical.

All references and publications cited herein are expressly incorporated herein by reference in their entirety into this disclosure. Illustrative embodiments of this invention are discussed and reference has been made to possible variations within the scope of this invention. These and other variations and modifications in the invention will be apparent to those skilled in the art without departing from the scope of the invention, and it should be understood that this invention is not limited to the illustrative embodiments set forth herein. Accordingly, the invention is to be limited only by the claims provided below and equivalents thereof.

1. A sectioning blade comprising:
a blade body comprising a straight cutting edge, a back edge located opposite the straight cutting edge, a first end extending from the back edge to the straight cutting edge, and a second end located opposite the first end and extending from the back edge to the straight cutting edge;
wherein the first end comprises a first curved segment proximate the straight cutting edge and a first end segment proximate the back edge, wherein the first curved segment and the first end segment meet at a location intermediate the straight cutting edge and the back edge such that the first curved segment and the first end segment extend from the straight cutting edge to the back edge of the blade body;
and wherein the second end comprises a second curved segment proximate the straight cutting edge and a second end segment proximate the back edge, wherein the second curved segment and the second end segment meet at a location intermediate the straight cutting edge and the back edge such that the second curved segment and the second end segment extend from the straight cutting edge to the back edge of the blade body;
and further wherein the first curved segment comprises a curved cutting edge.

2. A sectioning blade according to claim 1, wherein the second curved segment comprises a curved cutting edge.

3. A sectioning blade according to claim 1, wherein the first end segment is straight and wherein the second end segment is straight.

4. A sectioning blade according to claim 1, wherein the back edge is generally aligned with the straight cutting edge.

5. A sectioning blade according to claim 1, wherein the first end segment is straight and the second end segment is straight, and wherein the back edge is generally aligned with the straight cutting edge.

6. A sectioning blade according to claim 1, wherein the first end segment is generally aligned with the second end segment, and wherein the first and second end segments are generally transverse to the straight cutting edge.

7. A sectioning blade according to claim 1, wherein the first end segment length is at least about one-half of the distance between the straight cutting edge and the back edge at the first end.

8. A sectioning blade according to claim 1, wherein the first end segment length is at least about one-half of the distance between the straight cutting edge and the back edge at the first end, and wherein the second end segment length is at least about one-half of the distance between the straight cutting edge and the back edge at the second end.

9. A sectioning blade according to claim 1, wherein the first and second curved segments are extend over an arc of at most about ninety degrees.

10. A sectioning blade according to claim 1, wherein the first and second curved segments are contiguous with the straight cutting edge.

11. A sectioning blade according to claim 1, wherein the blade body comprises one or more holes and a blade handle, wherein the blade handle comprises one or more posts extending through the one or more holes of the blade body when removably attached to the blade body.

12. A sectioning blade comprising:
a blade body comprising a straight cutting edge, a back edge located opposite the straight cutting edge, a first end extending from the back edge to the straight cutting edge, and a second end located opposite the first end and extending from the back edge to the straight cutting edge;
wherein the first end comprises a first curved segment proximate the straight cutting edge and a first end segment proximate the back edge, wherein the first curved segment and the first end segment meet at a location intermediate the straight cutting edge and the back edge such that the first curved segment and the first end segment extend from the straight cutting edge to the back edge of the blade body;
wherein the second end comprises a second curved segment proximate the straight cutting edge and a second end segment proximate the back edge, wherein the second curved segment and the second end segment meet at a location intermediate the straight cutting edge and the back edge such that the second curved segment and the second end segment extend from the straight cutting edge to the back edge of the blade body;
wherein the first curved segment comprises a curved cutting edge and the second curved segment comprises a curved cutting edge;
and wherein the first end segment length is at least about one-half of the distance between the straight cutting edge and the back edge at the first end.

13. A sectioning blade according to claim 12, wherein the first end segment length is at least about one-half of the distance between the straight cutting edge and the back edge at the first end.

14. A sectioning blade according to claim 12, wherein the first end segment length is at least about one-half of the distance between the straight cutting edge and the back edge at the first end, and wherein the second end segment length is at least about one-half of the distance between the straight cutting edge and the back edge at the second end.

15. A sectioning blade according to claim 12, wherein the first and second curved segments are extend over an arc of at most about ninety degrees.

16. A sectioning blade according to claim 12, wherein the first and second curved segments are contiguous with the straight cutting edge.

17. A sectioning blade comprising:
a blade body comprising a straight cutting edge, a back edge located opposite the straight cutting edge, a first end extending from the back edge to the straight cutting edge, and a second end located opposite the first end and extending from the back edge to the straight cutting edge;
wherein the first end comprises a first curved segment proximate the straight cutting edge and a first end segment proximate the back edge, wherein the first curved segment and the first end segment meet at a location intermediate the straight cutting edge and the back edge such that the first curved segment and the first end segment extend from the straight cutting edge to the back edge of the blade body;
and wherein the second end comprises a second curved segment proximate the straight cutting edge and a second end segment proximate the back edge, wherein the second curved segment and the second end segment meet at a location intermediate the straight cutting edge and the back edge such that the second curved segment and the second end segment extend from the straight cutting edge to the back edge of the blade body;
and further wherein the first curved segment comprises a curved cutting edge;
wherein the first and second curved segments are extend over an arc of at most about ninety degrees;
and wherein the first and second curved segments are contiguous with the straight cutting edge.

18. A sectioning blade according to claim 17, wherein the second curved segment comprises a curved cutting edge.

19. A sectioning blade according to claim 17, wherein the first end segment length is at least about one-half of the distance between the straight cutting edge and the back edge at the first end.

20. A sectioning blade according to claim 17, wherein the first end segment length is at least about one-half of the distance between the straight cutting edge and the back edge at the first end, and wherein the second end segment length is at least about one-half of the distance between the straight cutting edge and the back edge at the second end.
